(19) United States
(12) Patent Application Publication
(10) Pub. No.: US 2012/0028992 A1
(43) Pub. Date: Feb. 2, 2012

(54) Pharmaceutical compositions for terminating acute episodes of cardiac arrhythmia, restoring sinus rhythm, preventing recurrence of cardiac arrhythmia and/or maintaining normal sinus rhythm in mammals

(76) Inventor: Arthur M. Brown, Cleveland, OH (US)

(21) Appl. No.: 12/805,460

(22) Filed: Aug. 2, 2010

(51) Int. Cl. A61K 31/495; A61P 9/06

(52) U.S. Cl. 514/255.04

(57) ABSTRACT

Disclosed are pharmaceutical compositions for and methods of preventing or treating acute and/or chronic cardiac arrhythmias in a mammal, including terminating acute episodes of cardiac arrhythmia, restoring normal sinus rhythm, preventing recurrence of cardiac arrhythmia and/or maintaining normal sinus rhythm, which contain a combination of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine.

### BACKGROUND OF THE INVENTION

1. Field of the Invention

The present invention relates to pharmaceutical compositions for terminating acute episodes of cardiac arrhythmia, such as atrial fibrillation or ventricular fibrillation, in a mammal, such as a human, particularly pharmaceutical compositions containing 1-[2-[(4-fluorophenyl)methoxy]ethyl]-4-(3-phenylpropyl)piperazine and a pharmaceutically acceptable carrier. The present invention also relates to pharmaceutical compositions for maintaining sinus rhythm in a mammal, such as a human, and so preventing a recurrence of an episode of cardiac arrhythmia in that mammal, particularly pharmaceutical compositions containing 1-[2-[(4-fluorophenyl)methoxy]ethyl]-4-(3-phenylpropyl)piperazine and a pharmaceutically acceptable carrier.

2. Background of the Related Art

Atrial flutter and/or atrial fibrillation (AF) are the most commonly sustained cardiac arrhythmias in clinical practice, and are likely to increase in prevalence with the aging of the population. Currently, AF affects more than 1 million Americans annually, represents over 5% of all admissions for cardiovascular diseases and causes more than 80,000 strokes each year in the United States. While AF is rarely a lethal arrhythmia, it is responsible for substantial morbidity and can lead to complications such as the development of congestive heart failure or thromboembolism. Currently available Class I and Class III anti-arrhythmic drugs reduce the rate of recurrence of AF, but are of limited use because of a variety of potentially adverse effects, including ventricular proarrhythmia. Because current therapy is inadequate and fraught with side effects, there is a clear need to develop new therapeutic approaches.

Ventricular fibrillation (VF) is the most common cause associated with acute myocardial infarction, ischemic coronary artery disease and congestive heart failure. As with AF, current therapy is inadequate and there is a need to develop new therapeutic approaches.

Although various anti-arrhythmic agents are now available on the market, those having both satisfactory efficacy and a high margin of safety have not been obtained. For example, anti-arrhythmic agents of Class I, according to the classification scheme of Vaughan-Williams (“Classification of antiarrhythmic drugs,” Cardiac Arrhythmias, edited by: E. Sandoe, E. Flensted-Jensen, K. Olesen; Sweden, Astra, Sodertalje, pp 449-472 (1981)), which cause a selective inhibition of the maximum velocity of the upstroke of the action potential (Vₘₐₓ) are inadequate for preventing ventricular fibrillation because they shorten the wave length of the cardiac action potential, thereby favoring re-entry. In addition, they have problems regarding safety, i.e. they cause a depression of myocardial contractility and have a tendency to induce arrhythmias due to an inhibition of impulse conduction. The CAST (coronary artery suppression trial) study was terminated while in progress because the Class I antagonists had a higher mortality than placebo controls. β-adrenergenic receptor blockers and calcium channel (I_{Ca}) antagonists, which belong to Class II and Class IV, respectively, have a defect in that their effects are either limited to a certain type of arrhythmia or are contraindicated because of their cardiac depressant properties in certain patients with cardiovascular disease. Their safety, however, is higher than that of the anti-arrhythmic agents of Class I.

Anti-arrhythmic agents of Class III are drugs that cause a selective prolongation of the action potential duration (APD) without a significant depression of the maximum upstroke velocity (Vmax). They therefore lengthen the save length of the cardiac action potential increasing refractories, thereby antagonizing re-entry. Available drugs in this class are limited in number. Examples such as sotalol and amiodarone have been shown to possess interesting Class III properties (Singh B. N., Vaughan Williams E. M., “A third class of anti-arrhythmic action: effects on atrial and ventricular intracellular potentials and other pharmacological actions on cardiac muscle of MJ 1999 and AH 3747”, Br. J. Pharmacol 39:675-689 (1970), and Singh B. N., Vaughan Williams E. M., “The effect of amiodarone, a new anti-anginal drug, on cardiac muscle”, Br. J. Pharmacol 39:657-667 (1970)), but these are not selective Class III agents.

Sotalol also possesses Class II (β-adrenergic blocking) effects which may cause cardiac depression and is contraindicated in certain susceptible patients.

Amiodarone also is not a selective Class III antiarrhythmic agent because it possesses multiple electrophysiological actions and is severely limited by side effects. (Nademanee, K., “The Amiodarone Odyssey”, J. Am. Coll. Cardiol. 20:1063-1065 (1992)) Drugs of this class are expected to be effective in preventing ventricular fibrillation. Selective Class III agents, by definition, are not considered to cause myocardial depression or an induction of arrhythmias due to inhibition of conduction of the action potential as seen with Class I antiarrhythmic agents.

Class III agents increase myocardial refractoriness via a prolongation of cardiac action potential duration (APD). Theoretically, prolongation of the cardiac action potential can be achieved by enhancing inward currents (i.e. Na⁺ or Ca²⁺ currents; hereinafter I_{Na} and I_{Ca}, respectively) or by reducing outward repolarizing potassium K⁺ currents. The delayed rectifier (I_{K}) K⁺ current is the main outward current involved in the overall repolarization process during the action potential plateau, whereas the transient outward (Iₜₒ) and inward rectifier (I_{KI}) K⁺ currents are responsible for the rapid initial and terminal phases of repolarization, respectively. Cellular electrophysiologic studies have demonstrated that I_{K} consists of two pharmacologically and kinetically distinct K⁺ current subtypes, I_{Kr} (rapidly activating and deactivating) and I_{Ks} (slowly activating and deactivating). (Sanguinetti and Jurkiewicz, “Two components of cardiac delayed rectifier K⁺ current. Differential sensitivity to block by Class III anti-arrhythmic agents,” J Gen Physiol 96:195-215 (1990)). I_{Kr} is also the product of the human ether-a-go-go gene (hERG). Expression of hERG cDNA in cell lines leads to production of the hERG current which is almost identical to I_{Kr} (Curran et al, “A molecular basis for cardiac arrhythmia: hERG mutations cause long QT syndrome,” Cell 80(5):795-803 (1995)).

Class III anti-arrhythmic agents currently in development, including d-sotalol, dofetilide (UK-68,798), almokalant (H234/09), E-4031 and methanesulfonamide-N-[1′-6-cyano-1,2,3,4-tetrahydro-2-naphthalenyl)-3,4-dihydro-4-hydroxyspiro[2H-1-benzopyran-2,4′-piperidin]-6yl], (+)-, monochloride (MK-499) predominantly, if not exclusively, block IKr. Although, amiodarone is a blocker of I_{Ks} (Balser J. R. Bennett, P. B., Hondeghem, L. M. and Roden, D. M. “Suppression of time-dependent outward current in guinea pig ventricular myocytes: Actions of quinidine and amiodarone,” Circ. Res. 69:519-529 (1991)), it also blocks I_{Na} and I_{Ca}, effects thyroid function, is as a nonspecific adrenergic blocker, acts as an inhibitor of the enzyme phospholipase, and causes pulmonary fibrosis (Nademanee, K. “The Amiodarone Odessey,” J. Am. Coll. Cardiol. 20:1063-1065 (1992)).

Reentrant excitation (reentry) has been shown to be a prominent mechanism underlying supraventricular arrhythmias in man. Reentrant excitation requires a critical balance between slow conduction velocity and sufficiently brief refractory periods to allow for the initiation and maintenance of multiple reentry circuits to coexist simultaneously and sustain AF. Increasing myocardial refractoriness by prolonging APD, prevents and/or terminates reentrant arrhythmias. Most selective, Class III antiarrhythmic agents currently in development, such as d-sotalol and dofetilide predominantly, if not exclusively, block I_{Kr}, the rapidly activating component of I_{K} found both in atrium and ventricle in man.

Since these I_{Kr} blockers increase APD and refractoriness both in atria and ventricle without affecting conduction per se, theoretically they represent potential useful agents for the treatment of arrhythmias like AF and VF. These agents have a liability in that they have an enhanced risk of proarrhythmia at slow heart rates. For example, torsade de pointes, a specific type of polymorphic ventricular tachycardia which is commonly associated with excessive prolongation of the electrocardigraphic QT interval, hence termed “acquired long QT syndrome,” has been observed when these compounds are utilized (Roden, D. M. “Current Status of Class III Antiarrhythmic Drug Therapy,” Am J. Cardiol, 72:44B-49B (1993)). The exaggerated effect at slow heart rates has been termed “reverse frequency-dependence” and is in contrast to frequency-independent or frequency-dependent actions. (Hondeghem, L. M., “Development of Class III Antiarrhythmic Agents,” J. Cardiovasc. Cardiol. 20 (Suppl. 2):S17-S22). The pro-arrhythmic tendency led to suspension of the SWORD trial when d-sotalol had a higher mortality than placebo controls.

The slowly activating component of the delayed rectifier (I_{Ks}) potentially overcomes some of the limitations of I_{Kr} blockers associated with ventricular arrhythmias. Because of its slow activation kinetics, however, the role of I_{Ks} in atrial repolarization may be limited due to the relatively short APD of the atrium. Consequently, although I_{Ks} blockers may provide distinct advantage in the case of ventricular arrhythmias, their ability to affect supra-ventricular tachyarrhythmias (SVT) is considered to be minimal.

Another major defect or limitation of most currently available Class III anti-arrhythmic agents is that their effect increases or becomes more manifest at or during bradycardia or slow heart rates, and this contributes to their potential for proarrhythmia. On the other hand, during tachycardia or the conditions for which these agents or drugs are intended and most needed, they lose most of their effect. This loss or diminishment of effect at fast heart rates has been termed “reverse use-dependence” (Hondeghem and Snyders, “Class III antiarrhythmic agents have a lot of potential but a long way to go: Reduced effectiveness and dangers of reverse use dependence,” Circulation, 81:686-690 (1990); Sadanaga et al., “Clinical evaluation of the use-dependent QRS prolongation and the reverse use-dependent QT prolongation of class III anti-arrhythmic agents and their value in predicting efficacy,” Amer. Heart Journal 126:114-121 (1993)), or “reverse rate-dependence” (Bretano, “Rate dependence of class III actions in the heart,” Fundam. Clin. Pharmacol. 7:51-59 (1993); Jurkiewicz and Sanguinetti, “Rate-dependent prolongation of cardiac action potentials by a methanesulfonanilide class III anti-arrhythmic agent: Specific block of rapidly activating delayed rectifier K+current by dofetilide,” Circ. Res. 72:75-83 (1993)). Thus, an agent that has a use-dependent or rate-dependent profile, opposite that possessed by most current class III anti-arrhythmic agents, should provide not only improved safety but also enhanced efficacy.

In view of the problems associated with current anti-arrhythmic agents, there remains a need for an effective treatment of cardiac arrhythmias in mammals, including terminating acute episodes of cardiac arrhythmia, restoring normal sinus rhythm, preventing recurrence of cardiac arrhythmia and/or maintaining normal sinus rhythm.

Each and every reference cited herein is hereby incorporated by reference in its entirety, where appropriate, for appropriate teachings of additional or alternative details, features and/or technical background.

### SUMMARY OF THE INVENTION

It is therefore an object of the present invention to provide pharmaceutical compositions for and methods of preventing or treating acute and/or chronic cardiac arrhythmias in a mammal, including terminating acute episodes of cardiac arrhythmia, restoring normal sinus rhythm, preventing recurrence of cardiac arrhythmia and/or maintaining normal sinus rhythm. Other objects, features and advantages of the present invention will be set forth in the detailed description of preferred embodiments that follows, and in part will be apparent from the description or may be learned by practice of the invention. These objects and advantages of the invention will be realized and attained by the compositions and methods particularly pointed out in the written description and claims hereof.

In accordance with these and other objects, the present invention provides a pharmaceutical composition which contains 1-[2-[(4-fluorophenyl)methoxy]ethyl]-4-(3-phenylpropyl)piperazine, and/or pharmaceutically acceptable salts and/or solvates thereof. In addition, the pharmaceutical composition further contains 1-[2-[bis(4-fluorophenyl)methoxy]ethyl]-4-(3-phenylpropyl)piperazine and/or pharmaceutically acceptable salts and/or solvates thereof.

In another embodiment of the present invention, a method is provided for treating acute and/or chronic cardiac arrhythmias in a mammal which comprises administering an effective amount of a combination of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine, and/or pharmaceutically acceptable salts and/or solvates of one or both thereof.

In yet another embodiment of the present invention, a method is provided for terminating acute episodes of cardiac arrhythmia in a mammal which comprises administering an effective amount of a combination of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine, and/or pharmaceutically acceptable salts and/or solvates of one or both thereof.

In yet another embodiment of the present invention, a method is provided for restoring normal sinus rhythm in a mammal which comprises administering an effective amount of a combination of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine, and/or pharmaceutically acceptable salts and/or solvates of one or both thereof.

In yet another embodiment of the present invention, a method is provided for preventing recurrence of cardiac arrhythmia in a mammal which comprises administering an effective amount of a combination of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine, and/or pharmaceutically acceptable salts and/or solvates of one or both thereof.

In yet another embodiment of the present invention, a method is provided for maintaining normal sinus rhythm in a mammal that has previously experience at least one episode of cardiac arrhythmia which comprises administering an effective amount of a combination of 1-[2-[(4-fluorophenyl)methoxy]ethyl]-4-(3-phenylpropyl)piperazine and 1-[2-[bis(4-fluorophenyl)methoxy]-ethyl]-4-(3-phenylpropyl)piperazine, and/or pharmaceutically acceptable salts and/or solvates of one or both thereof.

In yet another embodiment of the present invention, an article of manufacture is provided which comprises a package having deposited thereon a label describing the contents of the package and having deposited therein a pharmaceutical composition as described above in a suitable dosage form.

### DETAILED DESCRIPTION OF PREFERRED EMBODIMENTS

As used herein, the singular forms “a,” “an,” and “the” include the plural reference unless the context clearly dictates otherwise.

As used herein, the term “pharmaceutically acceptable” refers to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable benefit/risk ratio.

As used herein, “therapeutically effective amount” refers to an amount which is effective in reducing, eliminating, treating, preventing or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

As used herein, “unit dose” means a single dose which is capable of being administered to a subject, and which can be readily handled and packaged, remaining as a physically and chemically stable unit dose comprising either vanoxerine or a pharmaceutically acceptable composition comprising vanoxerine. Among the preferred embodiments of the present invention are methods of preventing and/or treating acute and/or chronic cardiac arrhythmias, such as atrial fibrillation and ventricular fibrillation, in a mammal, such as a human.

These preferred embodiments include pharmaceutical compositions which contain a combination of 1-[2-[(4-fluorophenyl)methoxy]ethyl]-4-(3-phenylpropyl)piperazine, and/or pharmaceutically acceptable salts and/or solvates thereof, and 1-[2-[bis(4-fluorophenyl)methoxy]ethyl]-4-(3-phenylpropyl)piperazine and/or pharmaceutically acceptable salts and/or solvates thereof.

These preferred embodiments also include a method for treating acute and/or chronic cardiac arrhythmias in a mammal which comprises administering an effective amount of a combination of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine, and/or pharmaceutically acceptable salts and/or solvates of one or both thereof.

These preferred embodiments also include a method for terminating acute episodes of cardiac arrhythmia in a mammal which comprises administering an effective amount of a combination of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine, and/or pharmaceutically acceptable salts and/or solvates thereof.

These preferred embodiments also include a method for restoring normal sinus rhythm in a mammal which comprises administering an effective amount of a combination of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine, and/or pharmaceutically acceptable salts and/or solvates thereof.

These preferred embodiments also include a method for preventing recurrence of cardiac arrhythmia in a mammal which comprises administering an effective amount of a combination of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine, and/or pharmaceutically acceptable salts and/or solvates thereof.

These preferred embodiments also include a method for maintaining normal sinus rhythm in a mammal that has previously experience at least one episode of cardiac arrhythmia which comprises administering an effective amount of a combination of 1-[2-[(4-fluorophenyl)methoxy]ethyl]-4-(3-phenylpropyl)piperazine and 1-[2-[bis(4-fluorophenyl)methoxy]-ethyl]-4-(3-phenylpropyl)piperazine, and/or pharmaceutically acceptable salts and/or solvates thereof.

1-[2-[(4-fluorophenyl)methoxy]ethyl]-4-(3-phenylpropyl)piperazine has the following chemical structure:

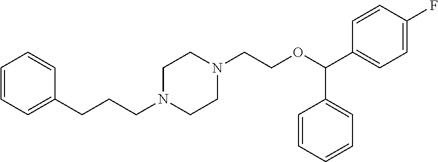

1-[2-[bis-(4-fluorophenyl)methoxy]ethyl]-4-(3-phenylpropyl)piperazine is also known as vanoxerine and has the following chemical structure:

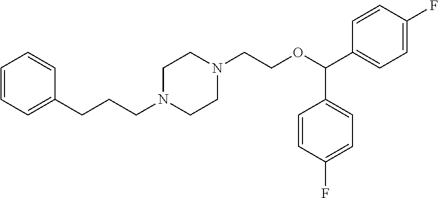

The manufacture and/or certain pharmaceutical uses of vanoxerine are described in U.S. Pat. No. 4,202,896, U.S. Pat. No. 4,476,129, U.S. Pat. No. 4,874,765, U.S. Pat. No. 6,743,797 and U.S. Pat. No. 7,700,600, as well as European Patent EP 243,903 and PCT International Application WO 91/01732, each of which is incorporated herein by reference in its entirety.

Pharmaceutically acceptable salts and/or solvates of 1-[2-[(4-fluorophenyl)methoxy]-ethyl]-4-(3-phenylpropyl)piperazine and/or 1-[2-[bis(4-fluorophenyl)methoxy]ethyl]-4-(3-phenylpropyl)piperazine may also be employed in the compositions and methods of the present invention.

The pharmaceutically acceptable salts which may be used include, but are not limited to, salts formed from non-toxic inorganic or organic acids. For example, pharmaceutically acceptable salts include, but are not limited to, the following: salts derived from inorganic acids, such as hydrochloric, hydrobromic, sulfuric, sulfamic, phosphoric, nitric and the like; salts derived from organic acids, such as acetic, propionic, succinic, glycolic, stearic, lactic, malic, tartaric, citric, ascorbic, pamoic, maleic, hydroxymaleic, phenylacetic, benzoic, salicylic, sulfanilic, 2-acetoxy-benzoic, fumaric, toluenesulfonic, methanesulfonic, ethane disulfonic, oxalic, isethionic, trifluoroacetic and the like; and salts derived from amino acids, such as glutamic acid or aspartic acid.

The pharmaceutically acceptable salts useful in the compositions and methods of the present invention can be synthesized from the parent compound by conventional chemical methods. Generally, the salts are prepared either by ion exchange chromatography or by reacting the free base with stoichiometric amounts or with an excess of the desired salt-forming inorganic or organic acid in a suitable solvent or various combinations of solvents.

When employed in the present methods, an effective amount of a combination of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine, or a pharmaceutically acceptable salt and/or solvate of one or both thereof, may be administered by any technique capable of introducing pharmaceutically active agent(s) to a desired site of action, including, but not limited to, buccal, sublingual, nasal, oral, topical, rectal and parenteral administration. Delivery of the compound may also be through the use of controlled release formulations in subcutaneous implants or transdermal patches.

Suitable doses of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine, or a pharmaceutically acceptable salt and/or solvate of one or both thereof, may be determined empirically by one skilled in the art depending upon such factors as the particular cardiac arrhythmias being treated (e.g. chronic or acute, atrial fibrillation or ventricular fibrillation, etc.), the species of mammal being treated (e.g. human), the physical characteristics of the mammal being treated (e.g. sex, weight, age, other physiological conditions, etc.) and the particular mode of administration being employed (e.g. oral, parenteral, etc.).

According to certain preferred embodiments, pharmaceutical compositions of the present invention contain a combination, exclusive of any excipients or carriers or other active agents, of from 0.01% to 1% of 1-[2-[(4-fluorophenyl)methoxy]ethyl]-4-(3-phenylpropyl)piperazine and from 99% to 99.99% of 1-[2-[bis(4-fluorophenyl)methoxy]ethyl]-4-(3-phenylpropyl)piperazine.

More preferably, pharmaceutical compositions of the present invention contain an combination containing from 0.05% to 1% of 1-[2-[(4-fluorophenyl)methoxy]ethyl]-4-(3-phenylpropyl)piperazine and even more preferably from 0.1% to 1% of 1-[2-[(4-fluorophenyl)methoxy]-ethyl]-4-(3-phenylpropyl)piperazine. Similarly, pharmaceutical compositions of the present invention contain a combination containing from 99% to 99.95% of 1-[2-[bis(4-fluorophenyl)methoxy]ethyl]-4-(3-phenylpropyl)piperazine and even more preferably from 99% to 99.9% of 1-[2-[bis(4-fluorophenyl)methoxy]ethyl]-4-(3-phenylpropyl)piperazine.

In preferred embodiments, a combination containing 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine generally comprises from about 20-50% by weight of the pharmaceutical composition, more preferably from about 25-40% and most preferably from about 30-35%.

Preferably, the inventive compositions also comprise: a diluent, such as lactose monohydrate; a binder, such as microcrystalline cellulose; a disintegrant, such as cross-linked sodium carboxymethyl cellulose; a flowing agent, such as colloidal silicon dioxide; and a lubricant, such as magnesium stearate. Suitable amounts of each excipient may be determined empirically by one skilled in the art considering such factors as the particular mode of administration (e.g. oral, sublingual, buccal, etc.), amount of active ingredient (e.g. 50 mg, 60 mg, 80 mg, 100 mg, 150 mg, etc.), particular patient (e.g. adult human, human child, etc.) and dosing regimen (e.g. once a day, twice a day, etc.).

In yet another embodiment of the present invention, an article of manufacture is provided which comprises a package having deposited thereon a label describing the contents of the package and having deposited therein one or more unit doses of a pharmaceutical composition as described above in a suitable form.

For oral administration, a suitable pharmaceutical composition may be prepared in the form of tablets, dragees, capsules, syrups and aqueous or oil suspensions. The inert ingredients used in the preparation of these compositions are known in the art. For example, tablets may be prepared by mixing the active compound with an inert diluent, such as lactose or calcium phosphate, in the presence of a disintegrating agent, such as potato starch or microcrystalline cellulose, and a lubricating agent, such as magnesium stearate or talc, and then tableting the mixture by known methods.

Tablets may also be formulated in a manner known in the art so as to give a sustained release of 1-[2-[(4-fluorophenyl)methoxy]ethyl]-4-(3-phenylpropyl)piperazine and/or 1-[2-[bis(4-fluorophenyl)methoxy]ethyl]-4-(3-phenylpropyl)piperazine. Such tablets may, if desired, be provided with enteric coatings by known method, for example by the use of cellulose acetate phthalate. Suitable binding or granulating agents are e.g. gelatine, sodium carboxymethylcellulose, methylcellulose, polyvinylpyrrolidone or starch gum. Talc, colloidal silicic acid, stearin as well as calcium and magnesium stearate or the like can be used as anti-adhesive and gliding agents.

Tablets may also be prepared by wet granulation and subsequent compression. A mixture containing 1-[2-[(4-fluorophenyl)methoxy]ethyl]-4-(3-phenylpropyl)piperazine and 1[2-[bis(4-fluorophenyl)methoxy]ethyl]-4-(3-phenylpropyl)piperazine and at least one diluent, and optionally a part of the disintegrating agent, is granulated together with an aqueous, ethanolic or aqueous-ethanolic solution of the binding agents in an appropriate equipment, then the granulate is dried. Thereafter, other preservative, surface acting, dispersing, disintegrating, gliding and anti-adhesive additives can be mixed to the dried granulate and the mixture can be compressed to tablets or capsules.

The tablets may also be prepared by the direct compression of the mixture containing the active ingredients together with the needed additives. If desired, the tablets may be transformed to dragees by using protective, flavoring and dyeing agents such as sugar, cellulose derivatives (methyl- or ethylcellulose or sodium carboxymethylcellulose), polyvinylpyrrolidone, calcium phosphate, calcium carbonate, food dyes, aromatizing agents, iron oxide pigments and the like which are commonly used in the pharmaceutical industry.

For the preparation of capsules or caplets, a mixture of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine and the desired additives may be filled into a capsule, such as a hard or soft gelatin capsule. The contents of a capsule and/or caplet may also be formulated using known methods to give sustained release of the active compound.

Liquid oral dosage forms may be an elixir, suspension and/or syrup, where the compound is mixed with a non-toxic suspending agent. Liquid oral dosage forms may also comprise one or more sweetening agent, flavoring agent, preservative and/or mixture thereof.

For rectal administration, a suitable composition may be prepared in the form of a suppository. In addition to the active ingredient, the suppository may contain a suppository mass commonly used in pharmaceutical practice, such as Theobroma oil, glycerinated gelatin or a high molecular weight polyethylene glycol.

For parenteral administration, a suitable composition may be prepared in the form of an injectable solution or suspension. For the preparation of injectable solutions or suspensions, the active ingredients can be dissolved in aqueous or non-aqueous isotonic sterile injection solutions or suspensions, such as glycol ethers, or optionally in the presence of solubilizing agents such as polyoxyethylene sorbitan monolaurate, monooleate or monostearate. These solutions or suspension may be prepared from sterile powders or granules having one or more carriers or diluents mentioned for use in the formulations for oral administration. Parenteral administration may be through intravenous, intradermal, intramuscular or subcutaneous injections.

A composition containing 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine may also be administered nasally, for example by sprays, aerosols, nebulised solutions and/or powders. Metered dose systems known to those in the art may also be used.

Pharmaceutical compositions may be administered to the buccal cavity (for example, sublingually) in known pharmaceutical forms for such administration, such as slow dissolving tablets, chewing gums, troches, lozenges, pastilles, gels, pastes, mouthwashes, rinses and/or powders.

A combination of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine may also be administered by continuous infusion either from an external source, for example by intravenous infusion or from a source of the compound placed within the body. Internal sources include implanted reservoirs which continuously release active(s) by osmosis and implants which may be (a) liquid such as a suspension or solution in a pharmaceutically acceptable oil of the compound(s) to be infused for example in the form of a very sparingly water-soluble derivative such as a dodecanoate salt or (b) solid in the form of an implanted support, for example of a synthetic resin or waxy material, for the compound to be infused. The support may be a single body containing all the compound or a series of several bodies each containing part of the compounds to be delivered. The amount of active should be such that a therapeutically effective amount is delivered over a long period of time.

In addition, an injectable solution of 1-[2-[(4-fluorophenyl)methoxy]ethyl]-4-(3-phenylpropyl)piperazine and 1-[2-[bis(4-fluorophenyl)methoxy]ethyl]-4-(3-phenylpropyl)piperazine can contain various additives such as preservatives, such as benzyl alcohol, methyl or propyl 4-hydroxybenzoate, benzalkonium chloride, phenylmercury borate and the like; as well as antioxidants, such as ascorbic acid, tocopherol, sodium pyrosulfate and optionally complex forming agents, such as an ethylenediamine tetraacetate salt for binding the metal traces, as well as buffers for adjusting the pH value and optionally a local anaesthetizing agent, e.g. lidocaine. The injectable solution is filtered before filling into the ampule and sterilized after filling.

Although the present invention has been described in considerable detail, those skilled in the art will appreciate that numerous changes and modifications may be made to the embodiments and preferred embodiments of the invention and that such changes and modifications may be made without departing from the spirit of the invention. It is therefore intended that the appended claims cover all equivalent variations as fall within the scope of the invention.

What is claimed is:

1. A pharmaceutical composition for preventing or treating acute and/or chronic cardiac arrhythmias in a mammal comprising an effective amount of a combination of 1-[2[(4-fluorophenyl)methoxy]ethyl]-4-(3-phenylpropyl)piperazine and 1-[2-[bis(4-fluorophenyl)methoxy]ethyl]-4-(3-phenylpropyl)piperazine.

2. The pharmaceutical composition of claim 1, further comprising at least one pharmaceutically acceptable excipient.

3. The pharmaceutical composition of claim 1, wherein said 1-[2-[(4-fluorophenyl)methoxy]ethyl]-4-(3-phenylpropyl)piperazine comprises from 0.01% to 1% of said composition and 1-[2-[bis(4-fluorophenyl)methoxy]ethyl]-4-(3-phenylpropyl)piperazine comprises from 99% to 99.99% of said composition.
